(19) United States
(12) Patent Application Publication
(10) Pub. No.: US 2020/0405753 A1
(43) Pub. Date: Dec. 31, 2020

(54) Oral Care Compositions and Methods of Use

(71) Applicant: Colgate-Palmolive Company, New York, NY (US)

(72) Inventors: Carlo DAEP, Brooklyn, NY (US); Paul THOMSON, Piscataway, NJ (US); Divino RAJAH, Long Valley, NJ (US)

(73) Assignee: Colgate-Palmolive Company, New York, NY (US)

(21) Appl. No.: 16/817,727

(22) Filed: Mar. 13, 2020

Related U.S. Application Data
(60) Provisional application No. 62/868,623, filed on Jun. 28, 2019.

(51) Int. Cl. A61K 33/30; A61K 33/16; A61K 9/00; A61Q 11/00; A61K 9/06; A61K 31/198; A61K 47/02; A61K 47/10; A61K 47/38

(52) U.S. Cl. CPC A61K 33/30; A61K 33/16; A61K 9/0053; A61Q 11/00; A61K 47/38; A61K 31/198; A61K 47/02; A61K 47/10; A61K 9/06

(57) ABSTRACT

The present disclosure relates to oral care compositions providing oral and/or systemic benefits and/or composed to facilitate recovery following oral surgery. In some embodiments, the oral care compositions of the present disclosure comprise one or more zinc ion sources (e.g., zinc oxide and zinc citrate) and a stannous ion source (e.g., stannous fluoride), and optionally an amino acid (e.g., arginine or a salt thereof), as well as to methods of making these compositions.

### FIELD

This invention relates to oral care compositions providing oral and/or systemic benefits and/or composed to facilitate recovery following oral surgery. In some embodiments, the oral care compositions of the present disclosure comprise one or more zinc ion sources (e.g., zinc oxide and zinc citrate) and a stannous ion source (e.g., stannous fluoride), and optionally an amino acid (e.g., arginine or a salt thereof), as well as to methods of making these compositions.

### BACKGROUND

Oral care compositions present particular challenges in preventing microbial contamination. Arginine and other basic amino acids have been proposed for use in oral care and are believed to have significant benefits in combating cavity formation and tooth sensitivity.

Commercially available arginine-based toothpaste for example, contains arginine bicarbonate and precipitated calcium carbonate, but not fluoride.

It has recently been recognized that oral infection (e.g., periodontitis) may affect the course and pathogenesis of a number of systemic diseases, such as endocarditis, cardiovascular disease, bacterial pneumonia, diabetes mellitus, and low birth weight. Various mechanisms linking oral infections to secondary systemic effects have been proposed, including metastatic spread of infection from the oral cavity as a result of transient bacteremia, metastatic injury from the effects of circulating oral microbial toxins, and metastatic inflammation caused by immunological injury induced by oral microorganisms. Bacterial infections of the oral cavity may affect the host's susceptibility to systemic disease in three ways: by shared risk factors; subgingival biofilms acting as reservoirs of gram-negative bacteria; and the periodontium acting as a reservoir of inflammatory mediators. Therefore, reducing the total biofilm load within the oral cavity would improve whole mouth health as well as support systemic health.

For example, a person may be particularly susceptible to deleterious effects stemming from bacterial presence within the oral cavity following dental procedures. Aside from the possibility of cross-infection within the dental facility, a patient who has undergone oral surgery oftentimes will have exposed wounds in the mouth while the treated area heals.

Certain types of bacteria known to dwell within the human oral cavity are understood to contribute to such systemic health issues. For example, Streptococcus gordonii are Gram-positive bacteria and are considered to be one of the initial colonizers of the oral cavity environment. The bacteria, along with other related oral streptococci and primary colonizing bacteria, have high affinity for molecules in the salivary pellicle coating the tooth surface therefore allowing the rapid colonization of a clean tooth surfaces. Oral streptococci ordinarily comprises the vast majority of the bacterial biofilm that forms on clean tooth surfaces. S. gordonii and related bacterial act as an attachment substrate for later colonizers of tooth surface, eventually facilitating the oral colonization of periodontal pathogens (e.g. Porphyromonas gingivitis and Fusobacterium nucleatum) via specific receptor-ligand interactions. Controlling plaque accumulation is important for gingival and oral health as well as contribute to improving the systemic well-being.

Endocarditis is an infection of the endocardium, the inner lining of the heart's chambers and valves. Endocarditis generally occurs when bacteria, fungi, or other pathogens from other body sites, including the mouth. Bacteria can infiltrate into oral tissues to reach the underlying network of blood vessels, eventually becoming systemically dispersed and colonize new sites for infection including the heart. If left unmanaged, endocarditis can lead to life-threatening complications. Treatments for endocarditis include antibiotics and, in certain cases, surgery.

Accordingly, there is a need for improved oral care compositions suitable for use in patients who are at risk for systemic bacterial infections. For example, there is a need for such oral care compositions to facilitate recovery following oral surgery, e.g., oral care compositions to reduce bacterial burden for the prevention of bacterial infections of soft tissue within the mouth of a susceptible patient population.

### BRIEF SUMMARY

It has been surprisingly found that oral care compositions comprising a zinc oxide and/or zinc citrate, a stannous ion source (e.g., stannous fluoride) and optionally an amino acid, (e.g., arginine), selected at certain concentrations and amounts, unexpectedly increases the antibacterial effect of oral care compositions, in the oral cavity of a user. The current formulations offer the advantage of robust microbial protection without significantly interfering with the stability of the oral care composition and by allowing for formulations which allow for the integration of a basic amino acid without compromising stannous and zinc availability and deposition in situ. The increased amount of available zinc and stannous aids in reducing bacterial viability, colonization, and biofilm development. Thus, the present compositions may be particularly useful in methods of treating or prophylaxis of gingivitis and, by relation, systemic bacterial infections stemming from oral bacteria and plaque accumulation.

Thus, in a first aspect, the present disclosure is directed to an oral care composition for use in the treatment or prophylaxis of a systemic bacterial infection consequent to promulgation of orally-derived bacteria, the oral care composition comprising at least one zinc ion source (e.g., zinc oxide and/or zinc citrate), a stannous ion source (e.g., stannous fluoride), and optionally a basic amino acid in free or salt from (e.g., free form arginine).

In a second aspect, the present disclosure is directed to a method of treatment or prophylaxis of a systemic bacterial infection consequent to promulgation of orally-derived bacteria, the method comprising use of an oral care composition comprising at least one zinc ion source (e.g., zinc oxide and/or zinc citrate), a stannous ion source (e.g., stannous fluoride), and optionally a basic amino acid in free or salt from (e.g., free form arginine).

### DETAILED DESCRIPTION

As used herein, the term “oral composition” means the total composition that is delivered to the oral surfaces. The composition is further defined as a product which, during the normal course of usage, is not, the purposes of systemic administration of particular therapeutic agents, intentionally swallowed but is rather retained in the oral cavity for a time sufficient to contact substantially all of the dental surfaces and/or oral tissues for the purposes of oral activity. Examples of such compositions include, but are not limited to, toothpaste or a dentifrice, a mouthwash or a mouth rinse, a topical oral gel, a denture cleanser, sprays, powders, strips, floss and the like.

As used herein, the term “dentifrice” means paste, gel, or liquid formulations unless otherwise specified. The dentifrice composition can be in any desired form such as deep striped, surface striped, multi-layered, having the gel surrounding the paste, or any combination thereof. Alternatively, the oral composition may be dual phase dispensed from a separated compartment dispenser.

In one aspect the invention is an oral care composition (Composition 1.0) for use in the treatment or prophylaxis of a systemic bacterial infection consequent to promulgation of orally-derived bacteria, in a subject in need thereof, the oral care composition comprising
a.) at least one zinc ion source (e.g., zinc oxide and/or zinc citrate) (e.g., zinc phosphate); and
b.) a stannous ion source (e.g., stannous fluoride)

For example, the invention contemplates any of the following compositions (unless otherwise indicated, values are given as percentage of the overall weight of the composition):

1.1. Any of the preceding compositions, wherein the zinc ion source is selected from zinc oxide, zinc citrate, zinc lactate, zinc phosphate and combinations thereof 1.2. Any of the preceding compositions, wherein the zinc ion source comprises or consists of a combination of zinc oxide and zinc citrate.

1.3. The preceding composition, wherein the ratio of the amount of zinc oxide (e.g., wt. %) to zinc citrate (e.g., wt %) is from 1.5:1 to 4.5:1 (e.g., 2:1, 2.5:1, 3:1, 3.5:1, or 4:1).

1.4. Either of the two preceding compositions, wherein the zinc citrate is in an amount of from 0.25 to 1.0 wt % (e.g., 0.5 wt. %) and zinc oxide may be present in an amount of from 0.75 to 1.25 wt % (e.g., 1.0 wt. %) based on the weight of the oral care composition.

1.5. Any of the preceding compositions, wherein the zinc ion source comprises zinc citrate in an amount of about 0.5 wt %.

1.6. Any of the preceding compositions, wherein the zinc ion source comprises zinc oxide in an amount of about 1.0 wt %.

1.7. Any of the preceding compositions, wherein the zinc ion source comprises zinc citrate in an amount of about 0.5 wt % and zinc oxide in an amount of about 1.0 wt %.

1.8. Any of preceding compositions wherein the composition is ethanol-free.

1.9. Any of the preceding compositions further comprising a fluoride source selected from: sodium fluoride, potassium fluoride, sodium monofluorophosphate, sodium fluorosilicate, ammonium fluorosilicate, amine fluoride (e.g., N′-octadecyltrimethylendiamine-N,N,N′-tris(2-ethanol)-dihydrofluoride), ammonium fluoride, titanium fluoride, hexafluorosulfate, and combinations thereof.

1.10. The preceding composition wherein the fluoride source is present in an amount of 0.1 wt. % to 2 wt. % (0.1 wt %-0.6 wt. %) of the total composition weight.

1.11. Any of the preceding compositions wherein the fluoride source provides fluoride ion in an amount of from 50 to 25,000 ppm (e.g., 750-7000 ppm, e.g., 1000-5500 ppm, e.g., about 500 ppm, 1000 ppm, 1100 ppm, 2800 ppm, 5000 ppm, or 25000 ppm).

1.12. Any of the preceding compositions wherein the pH is between 4.0 and 10.0, e.g., 5.0 to 8.0, e.g., 7.0 to 8.0.

1.13. Any of the preceding compositions further comprising calcium carbonate.

1.14. The preceding composition, wherein the calcium carbonate is a precipitated calcium carbonate high absorption (e.g., 20% to 30% by weight of the composition) (e.g., 25% precipitated calcium carbonate high absorption).

1.15. Any of the preceding compositions further comprising a precipitated calcium carbonate—light (e.g., about 10% precipitated calcium carbonate—light) (e.g., about 10% natural calcium carbonate).

1.16. Any of the preceding compositions further comprising an effective amount of one or more alkali phosphate salts, e.g., sodium, potassium or calcium salts, e.g., selected from alkali dibasic phosphate and alkali pyrophosphate salts, e.g., alkali phosphate salts selected from sodium phosphate dibasic, potassium phosphate dibasic, dicalcium phosphate dihydrate, calcium pyrophosphate, tetrasodium pyrophosphate, tetrapotassium pyrophosphate, sodium tripolyphosphate, disodium hydrogenorthophoshpate, monosodium phosphate, pentapotassium triphosphate and mixtures of any of two or more of these, e.g., in an amount of 0.01-20%, e.g., 0.1-8%, e.g., e.g., 0.1 to 5%, e.g., 0.3 to 2%, e.g., 0.3 to 1%, e.g. about 0.01%, about 0.1%, about 0.5%, about 1%, about 2%, about 5%, about 6%, by weight of the composition.

1.17. Any of the preceding compositions comprising tetrapotassium pyrophosphate, disodium hydrogenorthophoshpate, monosodium phosphate, and pentapotassium triphosphate.

1.18. Any of the preceding compositions comprising a polyphosphate.

1.19. The preceding composition, wherein the polyphosphate is tetrasodium pyrophosphate.

1.20. The preceding composition, wherein the tetrasodium pyrophosphate is from 0.1-1.0 wt % (e.g., about 0.5 wt %).

1.21. Any of the preceding compositions further comprising an abrasive or particulate (e.g., silica).

1.22. Any of the preceding compositions wherein the silica is synthetic amorphous silica. (e.g., 1%-28% by wt.) (e.g., 8%-25% by wt.)

1.23. The preceding composition, wherein the silica abrasives are silica gels or precipitated amorphous silicas, e.g. silicas having an average particle size ranging from 2.5 microns to 12 microns.

1.24. Any of the preceding compositions further comprising a small particle silica having a median particle size (d50) of 1-5 microns (e.g., 3-4 microns) (e.g., about 5 wt. % Sorbosil AC43 from PQ Corporation Warrington, United Kingdom).

1.25. Any of the three preceding compositions wherein 20-30 wt % of the total silica in the composition is small particle silica (e.g., having a median particle size (d50) of 3-4 microns) and wherein the small particle silica is about 5 wt. % of the oral care composition.

1.26. Any of the preceding compositions comprising silica wherein the silica is used as a thickening agent, e.g., particle silica.

1.27. Any of the preceding compositions further comprising a nonionic surfactant, wherein the nonionic surfactant is in an amount of from 0.5-5%, e.g., 1-2%, selected from poloxamers (e.g., poloxamer 407), polysorbates (e.g., polysorbate 20), polyoxyl hydrogenated castor oil (e.g., polyoxyl 40 hydrogenated castor oil), and mixtures thereof.

1.28. The preceding composition, wherein the poloxamer nonionic surfactant has a polyoxypropylene molecular mass of from 3000 to 5000 g/mol and a polyoxyethylene content of from 60 to 80 mol %, e.g., the poloxamer nonionic surfactant comprises poloxamer 407.

1.29. Any of the preceding compositions further comprising sorbitol, wherein the sorbitol is in a total amount of 10-40% (e.g., about 23%).

1.30. Any of the preceding compositions further comprising an additional ingredient selected from: benzyl alcohol, Methylisothizolinone (“MIT”), Sodium bicarbonate, sodium methyl cocoyl taurate (tauranol), lauryl alcohol, and polyphosphate.

1.31. Any of the preceding compositions comprising a flavoring, fragrance and/or coloring agent.

1.32. Any of the preceding compositions, wherein the composition further comprises a copolymer.

1.33. The preceding composition, wherein the copolymer is a PVM/MA copolymer.

1.34. The preceding composition, wherein the PVM/MA copolymer comprises a 1:4 to 4:1 copolymer of maleic anhydride or acid with a further polymerizable ethylenically unsaturated monomer; for example, 1:4 to 4:1, e.g. about 1:1.

1.35. The preceding composition, wherein the further polymerizable ethylenically unsaturated monomer comprises methyl vinyl ether (methoxyethylene).

1.36. Any of compositions 1.50-1.52, wherein the PVM/MA copolymer comprises a copolymer of methyl vinyl ether/maleic anhydride, wherein the anhydride is hydrolyzed following copolymerization to provide the corresponding acid.

1.37. Any of compositions 1.50-1.53, wherein the PVM/MA copolymer comprises a GANTREZ® polymer (e.g., GANTREZ® S-97 polymer).

1.38. Any of the preceding compositions, wherein the composition comprises a thickening agent selected from the group consisting of carboxyvinyl polymers, carrageenan, xanthan, hydroxyethyl cellulose and water soluble salts of cellulose ethers (e.g., sodium carboxymethyl cellulose and sodium carboxymethyl hydroxyethyl cellulose).

1.39. Any of the preceding compositions further comprising sodium carboxymethyl cellulose (e.g., from 0.5 wt. %-1.5 wt. %).

1.40. Any of the preceding compositions comprising from 5%-40%, e.g., 10%-35%, e.g., about 15%, 25%, 30%, and 35% water.

1.41. Any of the preceding compositions, wherein the stannous ion source is selected from stannous fluoride, other stannous halides such as stannous chloride dihydrate, stannous pyrophosphate, organic stannous carboxylate salts such as stannous formate, acetate, gluconate, lactate, tartrate, oxalate, malonate and citrate, stannous ethylene glyoxide, or a mixture thereof.

1.42. Any of the preceding compositions, wherein the stannous ion source comprises stannous fluoride

1.43. Any of the preceding compositions comprising an additional antibacterial agent selected from herbal extracts and essential oils (e.g., rosemary extract, tea extract, magnolia extract, thymol, menthol, eucalyptol, geraniol, carvacrol, citral, honokiol, catechol, methyl salicylate, epigallocatechin gallate, epigallocatechin, gallic acid, miswak extract, sea-buckthorn extract), bisguanide antiseptics (e.g., chlorhexidine, alexidine or octenidine), quaternary ammonium compounds (e.g., cetylpyridinium chloride (CPC), benzalkonium chloride, tetradecylpyridinium chloride (TPC), N-tetradecyl-4-ethylpyridinium chloride (TDEPC), phenolic antiseptics, hexetidine, octenidine, sanguinarine, povidone iodine, delmopinol, salifluor, metal ions (e.g., copper salts, iron salts), sanguinarine, propolis and oxygenating agents (e.g., hydrogen peroxide, buffered sodium peroxyborate or peroxycarbonate), phthalic acid and its salts, monoperthalic acid and its salts and esters, ascorbyl stearate, oleoyl sarcosine, alkyl sulfate, dioctyl sulfosuccinate, salicylanilide, domiphen bromide, delmopinol, octapinol and other piperidino derivatives, nicin preparations, chlorite salts; and mixtures of any of the foregoing.

1.44. Any of the preceding compositions comprising an antioxidant, e.g., selected from the group consisting of Co-enzyme Q10, PQQ, Vitamin C, Vitamin E, Vitamin A, BHT, anethole-dithiothione, and mixtures thereof.

1.45. Any of the preceding compositions comprising a whitening agent.

1.46. Any of the preceding compositions comprising a whitening agent selected from a whitening active selected from the group consisting of peroxides, metal chlorites, perborates, percarbonates, peroxyacids, hypochlorites, and combinations thereof.

1.47. Any of the preceding compositions further comprising hydrogen peroxide or a hydrogen peroxide source, e.g., urea peroxide or a peroxide salt or complex (e.g., such as peroxyphosphate, peroxycarbonate, perborate, peroxysilicate, or persulphate salts; for example, calcium peroxyphosphate, sodium perborate, sodium carbonate peroxide, sodium peroxyphosphate, and potassium persulfate), or hydrogen peroxide polymer complexes such as hydrogen peroxide-polyvinyl pyrrolidone polymer complexes.

1.48. Any of the preceding compositions comprising a basic amino acid (e.g., arginine)

1.49. Any of the preceding compositions, wherein the basic amino acid has the L-configuration (e.g., L-arginine).

1.50. Any of the preceding compositions, wherein the basic amino acid is arginine in free form.

1.51. Any of the preceding compositions wherein the basic amino acid is provided in the form of a di- or tri-peptide comprising arginine, or salts thereof.

1.52. Any of the preceding compositions wherein the basic amino acid is arginine, and wherein the arginine is present in an amount corresponding to 1% to 15%, e.g., 3 wt. % to 10 wt. % of the total composition weight, about e.g., 1.5%, 4%, 5%, or 8%, wherein the weight of the basic amino acid is calculated as free form.

1.53. Any of the preceding compositions wherein the amino acid is arginine from 0.1 wt. %-6.0 wt. %. (e.g., about 1.5 wt %).

1.54. Any of the preceding compositions wherein the amino acid is arginine from about 1.5 wt. %.

1.55. Any of the preceding compositions wherein the amino acid is arginine from 4.5 wt. %-8.5 wt. % (e.g., 5.0%)

1.56. Any of the preceding compositions wherein the amino acid is arginine from about 5.0 wt. %.

1.57. Any of the preceding compositions wherein the amino acid is arginine from 3.5 wt. %-9 wt. %.

1.58. Any of the preceding compositions wherein the amino acid is arginine from about 8.0 wt. %.

1.59. Any of the preceding compositions wherein the amino acid is L-arginine.

1.60. Any of the preceding compositions wherein the amino acid is arginine in partially or wholly in salt form.

1.61. Any of the preceding compositions wherein the amino acid is arginine phosphate.

1.62. Any of the preceding compositions wherein the amino acid is arginine hydrochloride.

1.63. Any of the preceding compositions wherein the amino acid is arginine bicarbonate.

1.64. Any of the preceding compositions wherein the amino acid is arginine ionized by neutralization with an acid or a salt of an acid.

1.65. Any of the preceding compositions further comprising an agent that interferes with or prevents bacterial attachment, e.g. ethyl lauroyl arginate (ELA) or chitosan.

1.66. Any of the preceding oral compositions, wherein the oral composition may be any of the following oral compositions selected from the group consisting of: a toothpaste or a dentifrice, a mouthwash or a mouth rinse, a topical oral gel, sprays, powders, strips, floss and a denture cleanser.

1.67. A composition obtained or obtainable by combining the ingredients as set forth in any of the preceding compositions.

1.68. Any of the preceding compositions, wherein the composition is for use in the treatment or prophylaxis of an oral and/or systemic bacterial infection involving the accumulation of biofilms of Gram negative bacterial interaction with Gram-positive bacteria (e.g., bacteria from the Streptococcus genus).

1.69. Any of the preceding compositions, wherein the composition is for use in the treatment or prophylaxis of an oral and/or systemic bacterial infection involving the accumulation of biofilms of Porphormonas gingivalis or Streptococcus gordonii.

1.70. Any of the preceding compositions, wherein the composition is for use in the treatment or prophylaxis of a systemic bacterial infection consequent to promulgation of a Gram negative bacterial interaction with Streptococcus gordonii.

1.71. Any of the preceding compositions, wherein the composition is for use in the treatment or prophylaxis of a systemic bacterial infection consequent to promulgation of orally-derived bacteria, selected from: gum disease (e.g., gingivitis or periodontitis), endocarditis (e.g., acute bacterial endocarditis), cardiovascular disease, bacterial pneumonia, diabetes mellitus, hardening of the aortic arch, circulatory deficiencies consequent to hardening of the aortic arch, increased blood pressures consequent to hardening of the aortic arch, and low birth weight.

1.72. Any of the preceding compositions, wherein the composition is for use in the treatment or prophylaxis systemic bacterial infection consequent to promulgation of orally-derived bacteria, selected from the group consisting of: endocarditis (e.g., acute bacterial endocarditis), cardiovascular disease, bacterial pneumonia, diabetes mellitus, hardening of the aortic arch, circulatory deficiencies consequent to hardening of the aortic arch, increased blood pressures consequent to hardening of the aortic arch, low birth weight

1.73. Any of the preceding compositions, wherein the composition is for use in the treatment or prophylaxis of endocarditis (e.g., acute bacterial endocarditis).

1.74. Any of the preceding compositions, wherein the composition is for use in the treatment or prophylaxis of an oral and/or systemic bacterial infection promulgated via transient bacteremia, metastatic injury from the effects of circulating oral microbial toxins, or metastatic inflammation caused by immunological injury induced by periodontal pathogens interaction with primary colonizing oral microorganisms (e.g., Streptococcus gordonii).

1.75. Any of the preceding compositions, wherein the composition is for use in the treatment or prophylaxis of endocarditis (e.g., acute bacterial endocarditis) promulgated via transient bacteremia metastatic injury from the effects of circulating oral microbial toxins, or metastatic inflammation caused by immunological injury induced by periodontal pathogens interaction with primary colonizing oral microorganisms (e.g., Streptococcus gordonii).

1.76. A composition obtained or obtainable by combining the ingredients as set forth in any of the preceding compositions.

1.77. A composition for use as set forth in any of the preceding compositions.

1.78 Any of the preceding compositions, wherein the zinc ion source comprises zinc phosphate (e.g., about 1.0 by wt) and wherein the stannous ion source is stannous fluoride.

The invention further comprises the use of sodium bicarbonate, sodium methyl cocoyl taurate (tauranol), MIT, and benzyl alcohol and combinations thereof in the manufacture of a Composition of the Invention, e.g., for use in any of the indications set forth in the above method of Composition 1.0, et seq.

### Methods of Use

In a second aspect, the present disclosure is directed to a method (Method 1) of treatment or prophylaxis of a disease or disorder related to an oral and/or systemic bacterial infection consequent to promulgation of orally-derived bacteria, to a subject in need thereof, the method comprising the administration of an oral care composition comprising:
a.) at least one zinc ion source (e.g., zinc oxide and/or zinc citrate) (e.g., zinc phosphate);
b.) a stannous ion source (e.g., stannous fluoride)

For example, the invention contemplates any of the following compositions (unless otherwise indicated, values are given as percentages of the overall weight of the composition):
1.1. Method 1, wherein the disease or disorder related to an oral and/or systemic bacterial infection consequent to the accumulation of biofilms of a Gram negative bacterial interaction with Gram-positive bacteria (e.g., bacteria from the Streptococcus genus).
1.2. Method 1 or 1.1, wherein the disease or disorder related to an oral and/or systemic bacterial infection consequent to the accumulation of biofilms of Porphormonas gingivalis and/or Streptococcus gordonii.
1.3. Any preceding method, wherein the disease or disorder related to a systemic bacterial infection consequent to promulgation of Streptococcus gordonii.
1.4. Any of the preceding methods, wherein the disease or disorder is gum disease (e.g., gingivitis or periodontitis), endocarditis (e.g., acute bacterial endocarditis), cardiovascular disease, bacterial pneumonia, diabetes mellitus, hardening of the aortic arch, circulatory deficiencies consequent to hardening of the aortic arch, increased blood pressures consequent to hardening of the aortic arch, low birth weight.
1.5. Any of the preceding methods, wherein the disease or disorder is endocarditis (e.g., acute bacterial endocarditis), cardiovascular disease, bacterial pneumonia, diabetes mellitus, hardening of the aortic arch, circulatory deficiencies consequent to hardening of the aortic arch, increased blood pressures consequent to hardening of the aortic arch, low birth weight.
1.6. Any of the preceding methods, wherein the disease or disorder is endocarditis (e.g., acute bacterial endocarditis).
1.7. Any of the preceding methods, wherein the disease or disorder related to a systemic bacterial infection is promulgated via transient bacteremia, metastatic injury from the effects of circulating oral microbial toxins, or metastatic inflammation caused by immunological injury induced by periodontal pathogens interaction with primary colonizing oral colonization of microorganisms.
1.8. Any of the preceding methods, wherein the disease or disorder is endocarditis (e.g., acute bacterial endocarditis) promulgated via transient bacteremia metastatic injury from the effects of circulating oral microbial toxins, or metastatic inflammation caused by periodontal pathogens interaction with primary colonizing immunological injury induced by oral microorganisms (e.g., Streptococcus gordonii).
1.9. Any of the preceding methods, comprising the step of applying the oral care composition to the oral cavity.
1.10. The preceding method, wherein the administration comprises brushing and/or rinsing a patient's teeth with the oral care dentifrice.
1.11. Any of the preceding methods, wherein the oral care composition is applied to a patient's teeth once, twice or three times daily.
1.12. Any of the preceding compositions, wherein the zinc ion source is selected from zinc oxide, zinc citrate, zinc lactate, zinc phosphate and combinations thereof.
1.13. Any of the preceding methods, wherein the zinc ion source comprises or consists of a combination of zinc oxide and zinc citrate.
1.14. Any of the preceding methods, wherein the zinc ion source comprises zinc phosphate.
1.15. Any of the preceding methods, wherein the ratio of the amount of zinc oxide (e.g., wt. %) to zinc citrate (e.g., wt %) is from 1.5:1 to 4.5:1 (e.g., 2:1, 2.5:1, 3:1, 3.5:1, or 4:1).
1.16. Any of the preceding methods, wherein the zinc citrate is in an amount of from 0.25 to 1.0 wt % (e.g., 0.5 wt. %) and zinc oxide may be present in an amount of from 0.75 to 1.25 wt % (e.g., 1.0 wt. %) based on the weight of the oral care composition.
1.17. Any of the preceding methods, wherein the zinc ion source comprises zinc citrate in an amount of about 0.5 wt %.
1.18. Any of the preceding methods, wherein the zinc ion source comprises zinc oxide in an amount of about 1.0 wt %.
1.19. Any of the preceding methods, wherein the zinc ion source comprises zinc citrate in an amount of about 0.5 wt % and zinc oxide in an amount of about 1.0 wt %.
1.20. Any of the preceding methods, wherein the zinc ion source comprises zinc phosphate in an amount of about 1.0 wt %.
1.21. Any of the preceding methods wherein the composition is ethanol-free.
1.22. Any of the preceding methods further comprising a fluoride source selected from: sodium fluoride, potassium fluoride, sodium monofluorophosphate, sodium fluorosilicate, ammonium fluorosilicate, amine fluoride (e.g., N′-octadecyltrimethylendiamine-N,N,N′-tris(2-ethanol)-dihydrofluoride), ammonium fluoride, titanium fluoride, hexafluorosulfate, and combinations thereof.
1.23. Any of the preceding methods wherein the fluoride source is present in an amount of 0.1 wt. % to 2 wt. % (0.1 wt %-0.6 wt. %) of the total composition weight.
1.24. Any of the preceding methods wherein the fluoride source provides fluoride ion in an amount of from 50 to 25,000 ppm (e.g., 750-7000 ppm, e.g., 1000-5500 ppm, e.g., about 500 ppm, 1000 ppm, 1100 ppm, 2800 ppm, 5000 ppm, or 25000 ppm).
1.25. Any of the preceding methods wherein the pH is between 4.0 and 10.0, e.g., 5.0 to 8.0, e.g., 7.0 to 8.0.
1.26. Any of the preceding methods further comprising calcium carbonate.
1.27. Any of the preceding methods, wherein the calcium carbonate is a precipitated calcium carbonate high absorption (e.g., 20% to 30% by weight of the composition) (e.g., 25% precipitated calcium carbonate high absorption).
1.28. Any of the preceding methods further comprising a precipitated calcium carbonate—light (e.g., about 10% precipitated calcium carbonate—light) (e.g., about 10% natural calcium carbonate).
1.29. Any of the preceding methods further comprising an effective amount of one or more alkali phosphate salts, e.g., sodium, potassium or calcium salts, e.g., selected from alkali dibasic phosphate and alkali pyrophosphate salts, e.g., alkali phosphate salts selected from sodium phosphate dibasic, potassium phosphate dibasic, dicalcium phosphate dihydrate, calcium pyrophosphate, tetrasodium pyrophosphate, tetrapotassium pyrophosphate, sodium tripolyphosphate, disodium hydrogenorthophoshpate, monosodium phosphate, pentapotassium triphosphate and mixtures of any of two or more of these, e.g., in an amount of 0.01-20%, e.g., 0.1-8%, e.g., e.g., 0.1 to 5%, e.g., 0.3 to 2%, e.g., 0.3 to 1%, e.g. about 0.01%, about 0.1%, about 0.5%, about 1%, about 2%, about 5%, about 6%, by weight of the composition.
1.30. Any of the preceding methods comprising tetrapotassium pyrophosphate, disodium hydrogenorthophoshpate, monosodium phosphate, and pentapotassium triphosphate.
1.31. Any of the preceding methods comprising a polyphosphate.
1.32. Any of the preceding methods, wherein the polyphosphate is tetrasodium pyrophosphate.
1.33. Any of the preceding methods, wherein the tetrasodium pyrophosphate is from 0.1-1.0 wt % (e.g., about 0.5 wt %).
1.34. Any of the preceding methods further comprising an abrasive or particulate (e.g., silica).
1.35. Any of the preceding methods wherein the silica is synthetic amorphous silica. (e.g., 1%-28% by wt.) (e.g., 8%-25% by wt.)
1.36. Any of the preceding methods, wherein the silica abrasives are silica gels or precipitated amorphous silicas, e.g. silicas having an average particle size ranging from 2.5 microns to 12 microns.
1.37. Any of the preceding methods further comprising a small particle silica having a median particle size (d50) of 1-5 microns (e.g., 3-4 microns) (e.g., about 5 wt. % Sorbosil AC43 from PQ Corporation Warrington, United Kingdom).
1.38. Any of the preceding methods wherein 20-30 wt % of the total silica in the composition is small particle silica (e.g., having a median particle size (d50) of 3-4 microns) and wherein the small particle silica is about 5 wt. % of the oral care composition.
1.39. Any of the preceding methods comprising silica wherein the silica is used as a thickening agent, e.g., particle silica.
1.40. Any of the preceding methods further comprising a nonionic surfactant, wherein the nonionic surfactant is in an amount of from 0.5-5%, e.g., 1-2%, selected from poloxamers (e.g., poloxamer 407), polysorbates (e.g., polysorbate 20), polyoxyl hydrogenated castor oil (e.g., polyoxyl 40 hydrogenated castor oil), and mixtures thereof.
1.41. Any of the preceding methods, wherein the poloxamer nonionic surfactant has a polyoxypropylene molecular mass of from 3000 to 5000 g/mol and a polyoxyethylene content of from 60 to 80 mol %, e.g., the poloxamer nonionic surfactant comprises poloxamer 407.
1.42. Any of the preceding methods further comprising sorbitol, wherein the sorbitol is in a total amount of 10-40% (e.g., about 23%).
1.43. Any of the preceding methods further comprising an additional ingredient selected from: benzyl alcohol, Methylisothizolinone (“MIT”), Sodium bicarbonate, sodium methyl cocoyl taurate (tauranol), lauryl alcohol, and polyphosphate.
1.44. Any of the preceding methods comprising a flavoring, fragrance and/or coloring agent.
1.45. Any of the preceding methods, wherein the composition further comprises a copolymer.
1.46. Any of the preceding methods, wherein the copolymer is a PVM/MA copolymer.
1.47. Any of the preceding methods, wherein the PVM/MA copolymer comprises a 1:4 to 4:1 copolymer of maleic anhydride or acid with a further polymerizable ethylenically unsaturated monomer; for example, 1:4 to 4:1, e.g. about 1:1.
1.48. Any of the preceding methods, wherein the further polymerizable ethylenically unsaturated monomer comprises methyl vinyl ether (methoxyethylene).
1.49. Any of the preceding methods 1.50-1.52, wherein the PVM/MA copolymer comprises a copolymer of methyl vinyl ether/maleic anhydride, wherein the anhydride is hydrolyzed following copolymerization to provide the corresponding acid.
1.50. Any of the preceding methods 1.50-1.53, wherein the PVM/MA copolymer comprises a GANTREZ® polymer (e.g., GANTREZ® S-97 polymer).
1.51. Any of the preceding methods, wherein the composition comprises a thickening agent selected from the group consisting of carboxyvinyl polymers, carrageenan, xanthan, hydroxyethyl cellulose and water soluble salts of cellulose ethers (e.g., sodium carboxymethyl cellulose and sodium carboxymethyl hydroxyethyl cellulose).
1.52. Any of the preceding methods further comprising sodium carboxymethyl cellulose (e.g., from 0.5 wt. %-1.5 wt. %).
1.53. Any of the preceding methods comprising from 5%-40%, e.g., 10%-35%, e.g., about 15%, 25%, 30%, and 35% water.
1.54. Any of the preceding methods, wherein the stannous ion source is selected from stannous fluoride, other stannous halides such as stannous chloride dihydrate, stannous pyrophosphate, organic stannous carboxylate salts such as stannous formate, acetate, gluconate, lactate, tartrate, oxalate, malonate and citrate, stannous ethylene glyoxide, or a mixture thereof.
1.55. Any of the preceding methods, wherein the stannous ion source comprises stannous fluoride
1.56. Any of the preceding methods comprising an additional antibacterial agent selected from herbal extracts and essential oils (e.g., rosemary extract, tea extract, magnolia extract, thymol, menthol, eucalyptol, geraniol, carvacrol, citral, honokiol, catechol, methyl salicylate, epigallocatechin gallate, epigallocatechin, gallic acid, miswak extract, sea-buckthorn extract), bisguanide antiseptics (e.g., chlorhexidine, alexidine or octenidine), quaternary ammonium compounds (e.g., cetylpyridinium chloride (CPC), benzalkonium chloride, tetradecylpyridinium chloride (TPC), N-tetradecyl-4-ethylpyridinium chloride (TDEPC), phenolic antiseptics, hexetidine, octenidine, sanguinarine, povidone iodine, delmopinol, salifluor, metal ions (e.g., copper salts, iron salts), sanguinarine, propolis and oxygenating agents (e.g., hydrogen peroxide, buffered sodium peroxyborate or peroxycarbonate), phthalic acid and its salts, monoperthalic acid and its salts and esters, ascorbyl stearate, oleoyl sarcosine, alkyl sulfate, dioctyl sulfosuccinate, salicylanilide, domiphen bromide, delmopinol, octapinol and other piperidino derivatives, nicin preparations, chlorite salts; and mixtures of any of the foregoing.
1.57. Any of the preceding methods comprising an antioxidant, e.g., selected from the group consisting of Co-enzyme Q10, PQQ, Vitamin C, Vitamin E, Vitamin A, BHT, anethole-dithiothione, and mixtures thereof.
1.58. Any of the preceding methods comprising a whitening agent.
1.59. Any of the preceding methods comprising a whitening agent selected from a whitening active selected from the group consisting of peroxides, metal chlorites, perborates, percarbonates, peroxyacids, hypochlorites, and combinations thereof.
1.60. Any of the preceding methods further comprising hydrogen peroxide or a hydrogen peroxide source, e.g., urea peroxide or a peroxide salt or complex (e.g., such as peroxyphosphate, peroxycarbonate, perborate, peroxysilicate, or persulphate salts; for example, calcium peroxyphosphate, sodium perborate, sodium carbonate peroxide, sodium peroxyphosphate, and potassium persulfate), or hydrogen peroxide polymer complexes such as hydrogen peroxide-polyvinyl pyrrolidone polymer complexes.
1.61. Any of the preceding methods comprising a basic amino acid (e.g., arginine)
1.62. Any of the preceding methods, wherein the basic amino acid has the L-configuration (e.g., L-arginine).
1.63. Any of the preceding methods, wherein the basic amino acid is arginine in free form.
1.64. Any of the preceding methods wherein the basic amino acid is provided in the form of a di- or tri-peptide comprising arginine, or salts thereof.
1.65. Any of the preceding methods wherein the basic amino acid is arginine, and wherein the arginine is present in an amount corresponding to 1% to 15%, e.g., 3 wt. % to 10 wt. % of the total composition weight, about e.g., 1.5%, 4%, 5%, or 8%, wherein the weight of the basic amino acid is calculated as free form.
1.66. Any of the preceding methods wherein the amino acid is arginine from 0.1 wt. % 6.0 wt. %. (e.g., about 1.5 wt %).
1.67. Any of the preceding methods wherein the amino acid is arginine from about 1.5 wt. %.
1.68. Any of the preceding methods wherein the amino acid is arginine from 4.5 wt. % 8.5 wt. % (e.g., 5.0%)
1.69. Any of the preceding methods wherein the amino acid is arginine from about 5.0 wt. %.
1.70. Any of the preceding methods wherein the amino acid is arginine from 3.5 wt. % 9 wt. %.
1.71. Any of the preceding methods wherein the amino acid is arginine from about 8.0 wt. %.
1.72. Any of the preceding methods wherein the amino acid is L-arginine.
1.73. Any of the preceding methods wherein the amino acid is arginine in partially or wholly in salt form.
1.74. Any of the preceding methods wherein the amino acid is arginine phosphate.
1.75. Any of the preceding methods wherein the amino acid is arginine hydrochloride.
1.76. Any of the preceding methods wherein the amino acid is arginine bicarbonate.
1.77. Any of the preceding methods wherein the amino acid is arginine ionized by neutralization with an acid or a salt of an acid.
1.78. Any of the preceding methods, wherein the oral care composition comprises an agent that interferes with or prevents bacterial attachment, e.g. ethyl lauroyl arginiate (ELA) or chitosan.
1.79. Any of the preceding methods, wherein the oral care composition may be any of the following oral compositions selected from the group consisting of: a toothpaste or a dentifrice, a mouthwash or a mouth rinse, a topical oral gel, sprays, powders, strips, floss and a denture cleanser.

The disclosure further provides an oral care composition for use in a method of treatment or prophylaxis of a systemic bacterial infection consequent to promulgation of orally-derived bacteria in a subject in need thereof, e.g., for use in any of Methods 1, et seq.

The disclosure further provides the use of an oral care composition in the manufacture of a medicament for the treatment or prophylaxis of a systemic bacterial infection consequent to promulgation of orally-derived bacteria, e.g., a medicament for use in any of Methods 1, et seq.

### Basic Amino Acids

The basic amino acids which can be used in the compositions and methods of the invention include not only naturally occurring basic amino acids, such as arginine, but also any basic amino acids having a carboxyl group and an amino group in the molecule, which are water-soluble and provide an aqueous solution with a pH of 7 or greater.

Accordingly, basic amino acids include, but are not limited to, arginine, serine, citrullene, ornithine, creatine, diaminobutanoic acid, diaminoproprionic acid, salts thereof or combinations thereof. In a particular embodiment, the basic amino acids are selected from arginine, citrullene, and ornithine.

In certain embodiments, the basic amino acid is arginine, for example, L-arginine, or a salt thereof.

The compositions of the invention are intended for topical use in the mouth and so salts for use in the present invention should be safe for such use, in the amounts and concentrations provided. Suitable salts include salts known in the art to be pharmaceutically acceptable salts are generally considered to be physiologically acceptable in the amounts and concentrations provided. Physiologically acceptable salts include those derived from pharmaceutically acceptable inorganic or organic acids or bases, for example acid addition salts formed by acids which form a physiological acceptable anion, e.g., hydrochloride or bromide salt, and base addition salts formed by bases which form a physiologically acceptable cation, for example those derived from alkali metals such as potassium and sodium or alkaline earth metals such as calcium and magnesium. Physiologically acceptable salts may be obtained using standard procedures known in the art, for example, by reacting a sufficiently basic compound such as an amine with a suitable acid affording a physiologically acceptable anion.

### Fluoride Ion Source

The oral care compositions may further include one or more fluoride ion sources, e.g., soluble fluoride salts. A wide variety of fluoride ion-yielding materials can be employed as sources of soluble fluoride in the present compositions. Examples of suitable fluoride ion-yielding materials are found in U.S. Pat. No. 3,535,421, to Briner et al.; U.S. Pat. No. 4,885,155, to Parran, Jr. et al. and U.S. Pat. No. 3,678,154, to Widder et al., each of which are incorporated herein by reference. Representative fluoride ion sources used with the present invention (e.g., Composition 1.0 et seq.) include, but are not limited to: stannous fluoride, sodium fluoride, potassium fluoride, sodium monofluorophosphate, sodium fluorosilicate, ammonium fluorosilicate, amine fluoride, ammonium fluoride, and combinations thereof. In certain embodiments the fluoride ion source includes: stannous fluoride, sodium fluoride, sodium monofluorophosphate as well as mixtures thereof. Where the formulation comprises calcium salts, the fluoride salts are preferably salts wherein the fluoride is covalently bound to another atom, e.g., as in sodium monofluorophosphate, rather than merely ionically bound, e.g., as in sodium fluoride.

### Surfactants

The invention may in some embodiments contain anionic surfactants, e.g., the Compositions of Composition 1.0, et seq., for example, water-soluble salts of higher fatty acid monoglyceride monosulfates, such as the sodium salt of the monosulfated monoglyceride of hydrogenated coconut oil fatty acids such as sodium N-methyl N-cocoyl taurate, sodium cocomo-glyceride sulfate; higher alkyl sulfates, such as sodium lauryl sulfate; higher alkyl-ether sulfates, e.g., of formula CH₃(CH₂)ₘCH₂(OCH₂CH₂)ₙOSO₃X, wherein m is 6-16, e.g., 10, n is 1-6, e.g., 2, 3 or 4, and X is Na or, for example sodium laureth-2 sulfate (CH₃(CH₂)₁₀CH₂(OCH₂CH₂)₂OSO₃Na); higher alkyl aryl sulfonates such as sodium dodecyl benzene sulfonate (sodium lauryl benzene sulfonate); higher alkyl sulfoacetates, such as sodium lauryl sulfoacetate (dodecyl sodium sulfoacetate), higher fatty acid esters of 1,2 dihydroxy propane sulfonate, sulfocolaurate (N-2-ethyl laurate potassium sulfoacetamide) and sodium lauryl sarcosinate. By “higher alkyl” is meant, e.g., C₆₋₃o alkyl. In particular embodiments, the anionic surfactant (where present) is selected from sodium lauryl sulfate and sodium ether lauryl sulfate. When present, the anionic surfactant is present in an amount which is effective, e.g., >0.001% by weight of the formulation, but not at a concentration which would be irritating to the oral tissue, e.g., 1%, and optimal concentrations depend on the particular formulation and the particular surfactant. In one embodiment, the anionic surfactant is present at from 0.03% to 5% by weight, e.g., 1.5%.

In another embodiment, cationic surfactants useful in the present invention can be broadly defined as derivatives of aliphatic quaternary ammonium compounds having one long alkyl chain containing 8 to 18 carbon atoms such as lauryl trimethylammonium chloride, cetyl pyridinium chloride, cetyl trimethylammonium bromide, di-isobutylphenoxyethyldimethylbenzylammonium chloride, coconut alkyltrimethylammonium nitrite, cetyl pyridinium fluoride, and mixtures thereof. Illustrative cationic surfactants are the quaternary ammonium fluorides described in U.S. Pat. No. 3,535,421, to Briner et al., herein incorporated by reference. Certain cationic surfactants can also act as germicides in the compositions.

Illustrative nonionic surfactants of Composition 1.0, et seq., that can be used in the compositions of the invention can be broadly defined as compounds produced by the condensation of alkylene oxide groups (hydrophilic in nature) with an organic hydrophobic compound which may be aliphatic or alkylaromatic in nature. Examples of suitable nonionic surfactants include, but are not limited to, the Pluronics, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and mixtures of such materials. In a particular embodiment, the composition of the invention comprises a nonionic surfactant selected from polaxamers (e.g., polaxamer 407), polysorbates (e.g., polysorbate 20), polyoxyl hydrogenated castor oils (e.g., polyoxyl 40 hydrogenated castor oil), betaines (such as cocamidopropylbetaine), and mixtures thereof.

Illustrative amphoteric surfactants of Composition 1.0, et seq., that can be used in the compositions of the invention include betaines (such as cocamidopropylbetaine), derivatives of aliphatic secondary and tertiary amines in which the aliphatic radical can be a straight or branched chain and wherein one of the aliphatic substituents contains about 8-18 carbon atoms and one contains an anionic water-solubilizing group (such as carboxylate, sulfonate, sulfate, phosphate or phosphonate), and mixtures of such materials.

The surfactant or mixtures of compatible surfactants can be present in the compositions of the present invention in 0.1% to 5%, in another embodiment 0.3% to 3% and in another embodiment 0.5% to 2% by weight of the total composition.

### Flavoring Agents

The oral care compositions of the invention may also include a flavoring agent. Flavoring agents which are used in the practice of the present invention include, but are not limited to, essential oils and various flavoring aldehydes, esters, alcohols, and similar materials, as well as sweeteners such as sodium saccharin. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Certain embodiments employ the oils of peppermint and spearmint.

The flavoring agent is incorporated in the oral composition at a concentration of 0.01 to 1% by weight.

### Chelating and Anti-Calculus Agents

The oral care compositions of the invention also may include one or more chelating agents able to complex calcium found in biofilm extrapolymeric substances (EPS). Binding of this calcium is believed to prevent biofilm calcification leading to prevention of calculus formation for better biofilm removal from the tooth surface.

Another group of agents suitable for use as chelating or anti-calculus agents in the present invention are the soluble pyrophosphates. The pyrophosphate salts used in the present compositions can be any of the alkali metal pyrophosphate salts. In certain embodiments, salts include tetra alkali metal pyrophosphate, di-alkali metal di-acid pyrophosphate, tri-alkali metal monoacid pyrophosphate and mixtures thereof, wherein the alkali metals are sodium or potassium. The salts are useful in both their hydrated and unhydrated forms. An effective amount of pyrophosphate salt useful in the present composition is generally enough to provide least 0.1 wt. % pyrophosphate ions, e.g., 0.1 to 3 wt 5, e.g., 0.1 to 2 wt %, e.g., 0.1 to 1 wt %, e.g., 0.2 to 0.5 wt %. The pyrophosphates also contribute to preservation of the compositions by lowering water activity.

### Polymers

The oral care compositions of the invention also optionally include one or more polymers, such as polyethylene glycols, polyvinyl methyl ether maleic acid copolymers, polysaccharides (e.g., cellulose derivatives, for example carboxymethyl cellulose, or polysaccharide gums, for example xanthan gum or carrageenan gum). Acidic polymers, for example polyacrylate gels, may be provided in the form of their free acids or partially or fully neutralized water soluble alkali metal (e.g., potassium and sodium) or ammonium salts. Certain embodiments include 1:4 to 4:1 copolymers of maleic anhydride or acid with another polymerizable ethylenically unsaturated monomer, for example, methyl vinyl ether (methoxyethylene) having a molecular weight (M.W.) of about 30,000 to about 1,000,000. These copolymers are available for example as Gantrez AN 139(M.W. 500,000), AN 1 19 (M.W. 250,000) and S-97 Pharmaceutical Grade (M.W. 70,000), of GAF Chemicals Corporation.

Other operative polymers include those such as the 1:1 copolymers of maleic anhydride with ethyl acrylate, hydroxyethyl methacrylate, N-vinyl-2-pyrollidone, or ethylene, the latter being available for example as Monsanto EMA No. 1 103, M.W. 10,000 and EMA Grade 61, and 1:1 copolymers of acrylic acid with methyl or hydroxyethyl methacrylate, methyl or ethyl acrylate, isobutyl vinyl ether or N-vinyl-2-pyrrolidone.

Suitable generally, are polymerized olefinically or ethylenically unsaturated carboxylic acids containing an activated carbon-to-carbon olefinic double bond and at least one carboxyl group, that is, an acid containing an olefinic double bond which readily functions in polymerization because of its presence in the monomer molecule either in the alpha-beta position with respect to a carboxyl group or as part of a terminal methylene grouping. Illustrative of such acids are acrylic, methacrylic, ethacrylic, alpha-chloroacrylic, crotonic, beta-acryloxy propionic, sorbic, alpha-chlorsorbic, cinnamic, beta-styrylacrylic, muconic, itaconic, citraconic, mesaconic, glutaconic, aconitic, alpha-phenylacrylic, 2-benzyl acrylic, 2-cyclohexylacrylic, angelic, umbellic, fumaric, maleic acids and anhydrides. Other different olefinic monomers copolymerizable with such carboxylic monomers include vinylacetate, vinyl chloride, dimethyl maleate and the like. Copolymers contain sufficient carboxylic salt groups for water-solubility.

A further class of polymeric agents includes a composition containing homopolymers of substituted acrylamides and/or homopolymers of unsaturated sulfonic acids and salts thereof, in particular where polymers are based on unsaturated sulfonic acids selected from acrylamidoalykane sulfonic acids such as 2-acrylamide 2 methylpropane sulfonic acid having a molecular weight of about 1,000 to about 2,000,000, described in U.S. Pat. No. 4,842,847, Jun. 27, 1989 to Zahid, incorporated herein by reference.

Another useful class of polymeric agents includes polyamino acids, particularly those containing proportions of anionic surface-active amino acids such as aspartic acid, glutamic acid and phosphoserine, as disclosed in U.S. Pat. No. 4,866,161 Sikes et al., incorporated herein by reference.

In preparing oral care compositions, it is sometimes necessary to add some thickening material to provide a desirable consistency or to stabilize or enhance the performance of the formulation. In certain embodiments, the thickening agents are carboxyvinyl polymers, carrageenan, xanthan gum, hydroxyethyl cellulose and water soluble salts of cellulose ethers such as sodium carboxymethyl cellulose and sodium carboxymethyl hydroxyethyl cellulose. Natural gums such as karaya, gum arabic, and gum tragacanth can also be incorporated. Colloidal magnesium aluminum silicate or finely divided silica can be used as component of the thickening composition to further improve the composition's texture. In certain embodiments, thickening agents in an amount of about 0.5% to about 5.0% by weight of the total composition are used.

### Abrasives

Natural calcium carbonate is found in rocks such as chalk, limestone, marble and travertine. It is also the principle component of egg shells and the shells of mollusks. The natural calcium carbonate abrasive of the invention is typically a finely ground limestone which may optionally be refined or partially refined to remove impurities. For use in the present invention, the material has an average particle size of less than 10 microns, e.g., 3-7 microns, e.g. about 5.5 microns. For example, a small particle silica may have an average particle size (D50) of 2.5-4.5 microns. Because natural calcium carbonate may contain a high proportion of relatively large particles of not carefully controlled, which may unacceptably increase the abrasivity, preferably no more than 0.01%, preferably no more than 0.004% by weight of particles would not pass through a 325 mesh. The material has strong crystal structure, and is thus much harder and more abrasive than precipitated calcium carbonate. The tap density for the natural calcium carbonate is for example between 1 and 1.5 g/cc, e.g., about 1.2 for example about 1.19 g/cc. There are different polymorphs of natural calcium carbonate, e.g., calcite, aragonite and vaterite, calcite being preferred for purposes of this invention. An example of a commercially available product suitable for use in the present invention includes Vicron® 25-11 FG from GMZ.

Precipitated calcium carbonate is generally made by calcining limestone, to make calcium oxide (lime), which can then be converted back to calcium carbonate by reaction with carbon dioxide in water. Precipitated calcium carbonate has a different crystal structure from natural calcium carbonate. It is generally more friable and more porous, thus having lower abrasivity and higher water absorption. For use in the present invention, the particles are small, e.g., having an average particle size of 1-5 microns, and e.g., no more than 0.1%, preferably no more than 0.05% by weight of particles which would not pass through a 325 mesh. The particles may for example have a D50 of 3-6 microns, for example 3.8=4.9, e.g., about 4.3; a D50 of 1-4 microns, e.g. 2.2-2.6 microns, e.g., about 2.4 microns, and a D10 of 1-2 microns, e.g., 1.2-1.4, e.g. about 1.3 microns. The particles have relatively high water absorption, e.g., at least 25 g/100 g, e.g. 30-70 g/100 g. Examples of commercially available products suitable for use in the present invention include, for example, Carbolag® 15 Plus from Lagos Industria Quimica.

In certain embodiments the invention may comprise additional calcium-containing abrasives, for example calcium phosphate abrasive, e.g., tricalcium phosphate (Ca₃(PO₄)₂), hydroxyapatite (Ca₁₀(PO₄)₆(OH)₂), or dicalcium phosphate dihydrate (CaHPO₄. 2H₂O, also sometimes referred to herein as DiCal) or calcium pyrophosphate, and/or silica abrasives, sodium metaphosphate, potassium metaphosphate, aluminum silicate, calcined alumina, bentonite or other siliceous materials, or combinations thereof. Any silica suitable for oral care compositions may be used, such as precipitated silicas or silica gels. For example synthetic amorphous silica. Silica may also be available as a thickening agent, e.g., particle silica. For example, the silica can also be small particle silica (e.g., Sorbosil AC43 from PQ Corporation, Warrington, United Kingdom). However the additional abrasives are preferably not present in a type or amount so as to increase the RDA of the dentifrice to levels which could damage sensitive teeth, e.g., greater than 130.

### Water

Water is present in the oral compositions of the invention. Water, employed in the preparation of commercial oral compositions should be deionized and free of organic impurities. Water commonly makes up the balance of the compositions and includes 5% to 45%, e.g., 10% to 20%, e.g., 25-35%, by weight of the oral compositions. This amount of water includes the free water which is added plus that amount which is introduced with other materials such as with sorbitol or silica or any components of the invention. The Karl Fischer method is a one measure of calculating free water.

### Humectants

Within certain embodiments of the oral compositions, it is also desirable to incorporate a humectant to reduce evaporation and also contribute towards preservation by lowering water activity. Certain humectants can also impart desirable sweetness or flavor to the compositions. The humectant, on a pure humectant basis, generally includes 15% to 70% in one embodiment or 30% to 65% in another embodiment by weight of the composition.

Suitable humectants include edible polyhydric alcohols such as glycerine, sorbitol, xylitol, propylene glycol as well as other polyols and mixtures of these humectants. Mixtures of glycerine and sorbitol may be used in certain embodiments as the humectant component of the compositions herein.

### pH Adjusting Agents

In some embodiments, the compositions of the present disclosure contain a buffering agent. Examples of buffering agents include anhydrous carbonates such as sodium carbonate, sesquicarbonates, bicarbonates such as sodium bicarbonate, silicates, bisulfates, phosphates (e.g., monopotassium phosphate, dipotassium phosphate, tribasic sodium phosphate, sodium tripolyphosphate, phosphoric acid), citrates (e.g. citric acid, trisodium citrate dehydrate), pyrophosphates (sodium and potassium salts) and combinations thereof. The amount of buffering agent is sufficient to provide a pH of about 5 to about 9, preferable about 6 to about 8, and more preferable about 7, when the composition is dissolved in water, a mouthrinse base, or a toothpaste base. Typical amounts of buffering agent are about 5% to about 35%, in one embodiment about 10% to about 30%, in another embodiment about 15% to about 25%, by weight of the total composition.

The present invention in its method aspect involves applying to the oral cavity a safe and effective amount of the compositions described herein.

The compositions and methods according to the invention (e.g., Composition 1.0 et seq) can be incorporated into oral compositions for the care of the mouth and teeth such as toothpastes, transparent pastes, gels, mouth rinses, sprays and chewing gum.

As used throughout, ranges are used as shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls. It is understood that when formulations are described, they may be described in terms of their ingredients, as is common in the art, notwithstanding that these ingredients may react with one another in the actual formulation as it is made, stored and used, and such products are intended to be covered by the formulations described.

The following examples further describe and demonstrate illustrative embodiments within the scope of the present invention. The examples are given solely for illustration and are not to be construed as limitations of this invention as many variations are possible without departing from the spirit and scope thereof. Various modifications of the invention in addition to those shown and described herein should be apparent to those skilled in the art and are intended to fall within the appended claims.

### EXAMPLES

### Example 1

### Example 1—Metal Penetration and Retention Assays

Zinc and stannous penetration and retention in salivary biofilms were evaluated using a laboratory model with a continuous media flow. Sterile HAP-coated glass microscope slides were pre-incubated with individually collected saliva inoculum containing saliva and plaque-derived bacteria for two hours at 37° C. under an environment containing 5% CO2. The inoculated slides were then transferred into a drip-flow biofilm reactor (Biosurface Technologies Corporation, Bozeman, Mont., USA) and incubated at 37° C. The biofilms were cultured under a constant flow rate of 10 mL/hour of growth medium consisting of 0.55 g/L proteose peptone (BD), 0.29 g/L trypticase peptone, 0.15 g/L potassium chloride (Sigma-Aldrich, St. Louis, Mo., USA), 0.029 g/L cysteine-HCL, 0.29 g/L yeast extract, 1.46 g/L dextrose, and 0.72 g/L mucin. The medium was supplemented with sodium lactate (0.024%, final concentration) and hemin (0.0016 mg/mL, final concentration). The biofilms were cultured for a total of 10 days. The resulting biofilms were then treated with dentifrice slurry diluted in sterile deionized water [1:2 (w/w)] for two minutes. Following treatment, the biofilms were washed twice in sterile deionized water (five-minute intervals) and then placed back into the biofilm reactors, resuming biofilm culture as previously described. The treated biofilms were allowed to recover for approximately 12 hours. The resultant biofilms were harvested by flash-freezing in liquid nitrogen and excised from the glass slides while carefully maintaining their orientation.

The biofilms were stored at −80° C. until analyzed by imaging mass spectroscopy. Biofilm samples were analyzed by Protea Biosciences (Morgantown, W. Va., USA) using Bruker UltrafleXtreme MALDI TOF/TOF. The biofilms were cryosectioned at 16 μm thickness and placed on stainless steel MALDI targets. The biofilms were coated with sinapinic acid (10 mg/mL, at a flow rate of 30 μL/min for a total of 30 coats) and allowed to dry for 20 seconds prior to analysis. The biofilm samples were ablated at 200 laser shots per pixel at a spatial resolution of 50 μm using reflectron positive ion mode. Sample mass ranges of between 100-1000 Daltons were collected and the images visualized using Bruker Flex Imaging.

A concentration map analysis of the resulting MALDI-MS image qualitatively demonstrates that biofilms treated with stannous, zinc citrate, zinc oxide, and arginine toothpaste formulations have improved stannous and zinc delivery as compared with standard pastes containing only stannous and zinc, and no arginine.

TABLE 1

| | |
|---|---|
| Formulation 1 | Stannous Fluoride 0.454%, |
| | 1% ZnOxide, |
| | 0.5% ZnCitrate, |
| | 3% Sodium Tripolyphosphate, |
| | 3.2% Flavoring |
| Formulation 2 | Stannous Fluoride 0.454%, |
| | 1% ZnOxide, |
| | 0.5% ZnCitrate, |
| | 1.5% Arginine, |
| | 3.2% Flavoring |
| Formulation 3 | Stannous Fluoride, |
| | Zinc lactate |

### Example 2

Salivary biofilms cultured for a total of 5 days in McBain medium, changing the media twice daily. Biofilms are cultured for 1 day prior to treatment. The resulting biofilms are treated with toothpaste slurries (1:2 in water) twice daily at approximately 12 hour intervals for 3 days. Following treatment, the treated biofilms are washed with sterile deionized water prior to returning into fresh culture media. On the fifth day, the biofilms are treated once in toothpaste slurry, rinsed in deionized water, and allowed to recover for 3 hours in sterile deionized water at 37 degrees Celsius. After recovery, the biofilms are harvested by sonication and analyzed for bacterial viability via ATP quantification as described by the manufacturer (Promega). Bacterial viability is measured based on percent reduction relative to control (Fluoride only treated biofilm). Percent reductions are determined across 3 different experiments comprising of approximately 4 biofilms per experiment.

In comparison to the sodium fluoride treated toothpaste, dentifrices formulated with stannous fluoride and zinc delivered significant antibacterial performance with reductions in viability ranging from 31-57%.

The antibacterial performance of a toothpaste containing stannous fluoride, zinc oxide, and zinc citrate technology was enhanced versus stannous fluoride+zinc lactate toothpaste (39% vs 31% respectively). Comparatively, biofilms treated with stannous fluoride+zinc oxide and zinc citrate+arginine had the greatest reduction in viability at 57% relative the fluoride treated control. This was ˜18% greater in antibacterial performance compared against the stannous fluoride+zinc oxide and zinc citrate toothpaste.

### Example 3

Saliva-derived biofilms, cultured from three different individuals, are independently cultured in McBain media supplemented with 5 ug/ml hemin and 1 ug/ml vitamin K for a total of 24 hours at 37 C under 5% CO2. The biofilms are cultured vertically on HAP disks, changing the media daily. The biofilms are then treated with slurries of test toothpastes once daily for two minutes under constant agitation at 80 rpm. The treated biofilms are then rinsed with sterile dH2O for 5 minutes 2×'s for a total of 4 days. The biofilms are recovered in sterile water for 3 hours after the last treatment prior to biofilm harvest. The treated biofilms are collected in 0.75 mL of sterile water and sonicated for 2 minutes at 30 second intervals per side. The collected biofilms are analyzed via ATP quantification. Total biofilm mass is estimated using Syto9 staining and comparing total mass based on the untreated groups.

Relative to the sodium fluoride only toothpaste (which do not contain zinc phosphate or stannous fluoride), biofilms treated with stannous fluoride high water/zinc phosphate toothpaste demonstrate a relative reduction in total biofilms (P=0.029). For example, upon ATP quantification, biofilms treated with sodium fluoride only toothpaste demonstrate ˜450,000 relative luminescence units (RLU's) versus biofilms treated with stannous fluoride high water/zinc phosphate toothpaste that demonstrate ˜300,000 RLU's.

### Example 4

Samples are evaluated for stannous and zinc delivery using the VitroSkin system. The Vitro-skin is cut (IMS Inc., Portland, Me.) into uniform circles of a particular diameter. The exact diameter is necessary to calculate uptake per square centimeter. To remove the silicone coating, the Vitro-skin circles are rinsed (in bulk) quickly with hexanes (done 3×), and air dried to evaporate hexanes. The Vitro-skin is soaked in sterilized and cleared saliva for 3 hours in 50 mL Falcon tube. Use 2 mL of saliva per tissue, and the assays are performed in triplicate. The studied toothpaste is added into a 20-mL scintillation vial and placed into 37° C. incubator/shaker. A fresh slurry is prepared right before the uptake experiment by adding 4 mL of 37° C. water into the vial and vortexing until the paste breaks. The saliva is aspirated from the tube with Vitro-skin. 1 mL the fresh paste slurry is added and incubated for 10 minutes in 37° C. incubator/shaker (speed 45). The slurry is aspirated immediately, and rinsed 3-times with 5 mL of DI water for 10 seconds each. A vortex is used for rinsing. The tissue is transferred into a new polystyrene 50 mL Falcon tube. 1 mL of concentrated nitric acid is added to the tissue and incubated overnight. The tissue should dissolve completely. Enough DI water is added to fill it to 10 mL line, and then shaken well. The solution may look hazy but no filtration is necessary. The obtained level of tin or zinc (typically in ppm) must be multiplied by the total volume (10× in this case) to get μg of tin or zinc per tissue (U_{T}). The data from the assay is presented in Table 2.

TABLE 2

| | Total Sn delivered | Total Zn delivered |
|---|---|---|
| Test Composition | (±StDev (ppm)) | (±StDev (ppm)) |
| Formula A | 26.43 ± 1.5* | 97.4 ± 3.92** |
| (Stannous Fluoride 0.454%, | | |
| Zinc Phosphate 1.0%, ~18% | | |
| H₂O) | | |
| Formula B | 14.3 ± 0.2 | 33 + 1.31 |
| (0.32 Sodium Fluoride, | | |
| 1.5% sodium lauryl sulfate) | | |

Paired, parametric t-test was performed to determine the statistical difference in metal delivered for each group
*Indicates statistical significance (P < 0.0046) vs Sn delivered with formula 3
**Indicates statistical significance (P < 0.0006) vs Zn delivered with formula 3

The following is a representative description of Formula described in Table 2

| Ingredient % w/w | Formulation A |
|---|---|
| Water | Q.S. |
| Stannous fluoride | 0.454 |
| Zinc phosphate, Hydrate | 1.0 |
| Thickeners | 2.8 |
| Sorbitol - Non-Crystal - 70% Soln USP, EP | 38.7 |
| Glycerin - USP, EP VEG | 6.0 |
| Abrasive silica | 10.0 |
| Trisodium Citrate Dihydrate | 3.0 |
| Sodium Carboxymethylcellulose | 0.8 |
| Polyethylene Glycol 600 | 2.0 |
| Tetrasodium Pyrophosphate | 2.0 |
| Anionic Surfactant | 1.5 |
| Amphoteric Surfactant | 1.25 |
| Anionic Polymer | 0.6 |
| Citric Acid - Anhydrous USP, EP | 0.6 |
| Flavors and Colors | 1.9 |
| Total Components | 100.0 |

While the present invention has been described with reference to embodiments, it will be understood by those skilled in the art that various modifications and variations may be made therein without departing from the scope of the present invention as defined by the appended claims.

1. A method of treatment or prophylaxis of a disease or disorder related to an oral and/or systemic bacterial infection consequent to promulgation of orally-derived bacteria, to a subject in need thereof, the method comprising the administration of an oral care composition comprising:
a.) at least one zinc ion source;
b.) a stannous ion source.

2. The method of claim 1, wherein the composition further comprises a basic amino acid.

3. The method of claim 2, wherein the basic amino acid is arginine in free form.

4. The method of claim 1, wherein the disease or disorder related to an oral and/or systemic bacterial infection consequent to the accumulation of biofilms of a Gram negative bacterial interaction with Gram-positive bacteria.

5. The method of claim 1, wherein the disease or disorder related to an oral and/or systemic bacterial infection consequent to the accumulation of biofilms of Porphormonas gingivalis and/or Streptococcus gordonii.

6. The method of claim 1, wherein the disease or disorder related to a systemic bacterial infection consequent to promulgation of Streptococcus gordonii.

7. The method of claim 1, wherein the disease or disorder is gum disease, endocarditis, cardiovascular disease, bacterial pneumonia, diabetes mellitus, hardening of the aortic arch, circulatory deficiencies consequent to hardening of the aortic arch, increased blood pressures consequent to hardening of the aortic arch, low birth weight.

8. The method of claim 1, wherein the disease or disorder is endocarditis, cardiovascular disease, bacterial pneumonia, diabetes mellitus, hardening of the aortic arch, circulatory deficiencies consequent to hardening of the aortic arch, increased blood pressures consequent to hardening of the aortic arch low, birth weight.

9. The method of claim 1, wherein the disease or disorder is endocarditis.

10. The method of claim 1, wherein the disease or disorder related to a systemic bacterial infection is promulgated via transient bacteremia, metastatic injury from the effects of circulating oral microbial toxins, or metastatic inflammation caused by immunological injury induced by periodontal pathogens interaction with primary colonizing oral colonization of microorganisms.

11. The method of claim 1, wherein the disease or disorder is endocarditis promulgated via transient bacteremia metastatic injury from the effects of circulating oral microbial toxins, or metastatic inflammation caused by periodontal pathogens interaction with primary colonizing immunological injury induced by oral microorganisms.

12. The method of claim 1, comprising the step of applying the oral care composition to the oral cavity.

13. The method of claim 1, wherein the administration comprises brushing and/or rinsing a patient's teeth with the oral care dentifrice.

14. The method of claim 1, wherein the oral care composition is applied to a patient's teeth once, twice or three times daily.

15. The method of claim 1, wherein the zinc ion source is selected from zinc oxide, zinc citrate, zinc lactate, zinc phosphate and combinations thereof.

16. The method of claim 15, wherein the zinc ion source comprises or consists of a combination of zinc oxide and zinc citrate.

17. The method of claim 16, wherein the ratio of the amount of zinc oxide to zinc citrate is from 1.5:1 to 4.5:1.

18. The method of claim 17, wherein the zinc citrate is in an amount of from 0.25 to 1.0 wt % and zinc oxide may be present in an amount of from 0.75 to 1.25 wt % based on the weight of the oral care composition.

19. (canceled)

20. (canceled)

21. The method of claim 1, wherein the stannous ion source is stannous fluoride.

22. The method of claim 15, wherein the zinc ion source comprises zinc phosphate.
